(19) United States
(12) Patent Application Publication
(10) Pub. No.: US 2019/0128781 A1
(43) Pub. Date: May 2, 2019

(54) ADAPTIVE AUTOMATED SAMPLING SYSTEM AND METHOD

(71) Applicants: Edward J Elliott, Tempe, AZ (US); Michael D Brown, Phoenix, AZ (US); Steve Stewart, Spring, TX (US)

(72) Inventors: Edward J Elliott, Tempe, AZ (US); Michael D Brown, Phoenix, AZ (US); Steve Stewart, Spring, TX (US)

(73) Assignee: OIL & GAS PROCESS SOLUTIONS, LLC, Phoenix, AZ (US)

(21) Appl. No.: 16/178,490

(22) Filed: Nov. 1, 2018

Related U.S. Application Data
(60) Provisional application No. 62/580,281, filed on Nov. 1, 2017.

(51) Int. Cl. G01N 1/20; G01N 33/28

(52) U.S. Cl. CPC G01N 1/2035; G01N 33/2823

(57) ABSTRACT

A hydrocarbon line sampler connected to a pipeline containing hydrocarbon fluid, the sampler including a draw mechanism and a sampling can. The sampler includes a detector for testing a quality attribute of the fluid. The detector passes a signal to a controller, the controller being preprogrammed with a set threshold of one or more quality thresholds. When a quality threshold is met, the controller can modify the sampling regimen for later storage or further on-site testing. The invention includes a method of drawing a sample, testing the sample, determining the quality of the sample, and modifying the sampling regimen as need, or otherwise setting an alarm, or taking action on the pipeline system.

### CLAIM OF PRIORITY

The present application includes subject matter disclosed in, and claims priority to, provisional application entitled “SMART ADAPTIVE AUTOMATED SAMPLING SYSTEM” filed Nov. 1, 2017 and assigned Ser. No. 62/580,281 describing an invention made by the present inventors and herein incorporated by reference.

### 1. FIELD OF THE INVENTION

The present invention relates to the field of measuring and testing fluid and more particularly, the present invention relates to controlled sample testing of fluid flow.

### 2. BACKGROUND OF THE INVENTION

To comply with Federal Energy Regulatory Commission (FERC) rules and in accordance with the American Petroleum Institute (API), product samples must be taken periodically of petroleum flowing through a pipeline. For example, it is known and required to take product samples of fluid for every 80 feet parcel of flowing product. In this instance, if there is a flow of 80 feet per second, a sample must be drawn every second. Multiple devices have been designed to accomplish this grabbing of samples from flowing petroleum products. Typical samples may be drawn of a volume of 1 to 50 cubic centimeters (cc). When multiple samples are taken in a single grabber or sampler, multiple receivers (typically bottles or cans (used herein interchangeably), may be used, one for each specific time period as is known in the art. Each sample take can also be known as a grab or bite or draw or sample. As known in art, spring-loaded piston or pneumatic pistons are often manually adjusted to set the amount of sample.

Present day pump volume regulators include pneumatic piston pumps and spring-loaded volume regulators. For instance, a present day volume analyzer can be driven by a pneumatic piston pump. A bite checker or volume analyzer monitors samples and pump volume. The device includes a single inlet and a single outlet functioning at atmospheric or low pressure. Similarly, overflow purge must travel into a low pressure, or atmospheric pressure, drain pan, tank, or sump. Pneumatic driven samplers provide no active control of the speed of the pump nor control of the volume. In effect, the device signals pump actuation and moves through an entire stroke at a speed determined by the power provided by the pneumatic system and friction/resistance therein.

It is common that multiple sources (e.g. resource fields, source pumps, etc.) will be aligned along a single pipeline. When a contaminant, or other unwanted property of the petroleum fluid, etc. is detected at a point in the line, it is not always so simple to determine the source of the contaminant. To compound the issue, by the time the detection is made, the petroleum may have traveled many miles, and may include inputs into the pipeline from multiple sources, over a long period of hours, or even days. By the time the contaminant is found, the fluid in the line is mixed to the point to an undiscernible level. There is not yet a system of pipeline or source line sampling that allows for the detection and identification of contaminated sources. Furthermore, current sampling systems are not sufficiently adaptive to alter pre-programmed sampling protocols if/when an issue is detected.

Therefore, there exists a need for manipulating the sample rate of a sampling.

It is therefore a primary object of the present invention to provide for an adaptive sampler system.

It is another object of the present invention to provide a remote controlled sampler system.

It is yet a further object of the present invention to provide a method for adaptive line sampling.

These and other objects of the present invention will become apparent to those skilled in the art as the description thereof proceeds.

### SUMMARY OF THE INVENTION

The sampler system of the present invention utilizes a controller-tied sampler for drawing samples of flow and moving such samples for storage and/or analysis such as into sampling receivers. The sampler of the present invention can work on a fast loop off of a main or directly, as is known in the art for sampling. The sampler is adaptable for direct crude sampling or direct pipeline draw, and can handle natural gas liquids (NGL) constant pressure applications, petroleum flows, or as are otherwise known to be required in the art.

The invention includes a sampling system for use with a source of hydrocarbon product. The system should include a controller adapted to receive a signal (either remote or local), and further capable of modifying an attribute of a sampling process based on the controller inputs. For instance, the controller can start/stop sampling, modify the frequency of samples taken, the size of samples taken, rate of pull, etc. An alarm may be attached to the sampler, the alarm may be able to alert remotely, possibly through a remote electronic signal. The sampler may have on-board testing equipment that can monitor for issues with the sampling, issues with the samples taken in receiver or in flow line, such as to detect water content in petroleum (referring generally to hydrocarbon in liquid form), and the controller can receive such information and modify the sampling scheme accordingly.

The present invention also includes a method of adaptive sampling of a flow of petroleum-based liquid. An inflow is connected to the sampling system to extract flowing petroleum-based liquid into a sampling system. A controller is attached, the controller capable of receiving inputs such as to the quality of the samples drawn, or from a remote station, and modifying the sampling scheme according to such inputs. In some cases, the controller receives direct communication from on-board testing system, and in other cases, it may receive a remote signal from off-site sampling testing (such as through a remote electronic or other signal). A local testing system may communicate to the controller directly, or via other communication means, such as Bluetooth, WiFi, etc. The controller may modify the outflow for petroleum-based fluid to exit the sampling system. For instance, the controller may start and/or stop sampling (modifying sampling time(s), lengths, etc.), may modify frequency, sampling rate, etc. The controller may also be attached to an alarm. The alarm may also be attached to a local testing system. The alarm can provide local notification of an issue (such as via sound, light, etc.). The alarm may also provide remote signaling of an event or condition of sampling, such as malfunction, or quality of samples/product taken. The alarm may also provide a distant communication of quality of sample to an operator to shut down a source or otherwise take further action.

### BRIEF DESCRIPTION OF THE DRAWINGS

The present invention will be described with greater specificity and clarity with reference to the following drawings, in which:

FIG. 1 illustrates a pipeline having a sampling system thereon;

FIG. 2 illustrates a pipeline including multiple inlets;

FIG. 3 illustrates a pipeline with multiple inlets including various sampling stations.

### DETAILED DESCRIPTION OF THE PREFERRED EMBODIMENT

The present invention includes a controlled and adaptive sampling system. Content of the drawn samples can be monitored and measured, and the controller can be programmed, reprogrammed, or automatically adapt to conditions of the draw (and/or quality attributes of hydrocarbon fluid) to modify the draw cycle and/or sample types taken. The controller can be remotely controlled and programmed to provide specified volumes, timing, and speed of sampling, location of sample, etc. Feedback and diagnostic systems can signal, report, or otherwise indicate a problem with the system. Furthermore, the system can include additional programming to compensate for sample condition discrepancies.

The present invention allows for line sampling modifications that may be triggered and reprogrammed. A smart automated adaptive sampler system can provide specific method/applications of modification to the sampling algorithm. The sampling can be modified by (i.e. how many seconds to draw, size and speed of draw to detect/determine contaminants, frequency adjustment, types/model of canisters (pressurized, warnings used, etc.)).

For instance, below is a list of specific input and action combination examples the new system may perform. This is just a few examples, and the specific variable quantities for illustrative purposes only. The number of possible combinations could be indefinite. For example, when testing determines of the sample:
If water cut measurement of tested sample exceeds 300 ppm and the flow rate is between 0 and 1000 barrels per hour (bbl/hr), modify sampling protocol to regimen of taking samples at a rate of 10 samples per minute.
If water cut measurement exceeds 300 ppm and the flow rate is between 1001 and 2500 (bbl/hr), start taking samples at a rate of 20 samples per minute.
If water cut measurement exceeds 500 ppm and the flow rate is between 0 and 2500 (bbl/hr), start taking 10 cc samples at a rate of 10 samples per minute.
If water cut measurement exceeds 1000 ppm and the flow rate is between 0 and 2500 (bbl/hr), start taking 20 cc samples at a rate of 10 samples per minute.
If viscosity measurement except 30 cPs, starting taking samples at rate of 1 cc sample every 10 bbl of flow on the line.
If H2S measurement exceeds 10 ppm, start taking samples of 2 cc every second and send and alarm signal to the control room.

One aspect of the present invention is the use of a programmable grabber that can take similar and/or controlled volume varied sample sizes in predetermined or programmable sequences. Testing/analyzing samples drawn may cause a signal to go to a processor. The processor determines if a predetermined threshold is met and modifies sampling process accordingly. When secondary (extreme) threshold is met, further alarm or action may be taken.

Petroleum products running through pipelines often must meet minimum standards for impurities and content. These specifications are monitored by a pipeline operator. In many instances, pipeline operators test samples that have origin from multiple sources. The present invention provides for multiple testing of the flow via samplers along the line. For instance, the sampler may be set after or between each supply input into the pipeline. In one embodiment, slip stream (bypass loop) may be used to run testing or samples that can provide input on the content/impurities of the product. The input from the testing stations, or input from an offsite testing station, can allow for local or remote reprogramming of a controller to modify testing. Input from local or remote testing may include issues with flow rate, density, water content, viscosity, optical color clarity, pressure, flashpoint, sulphur content, or other issues that can be or are currently tested in the art. Information can be added to the sampling system controller to modify testing via preset algorithms and/or manual overrides. Processor algorithm may be modified directly at sampler or remotely via e-communication.

Pressure and temperature readers and transmitters, temperature thermowells, test thermowells, and chromatography probes, ultrasonic meters, etc. can be used. A filter-separator may be sued before the before the sampler, or meter station, and may conduct continuous gas sampling. One method is to install a gas analyzer, to monitor gas for H2S, O2, N2, CO2 and other contamination, etc. that exceeds any specific quality requirements. If any agreed parameters for quality attributes are exceeded, the system is designed to shut down at chosen presets.

In practice, when an issue is determined to be present in the system, sampling will start and stop on a more frequent basis. For instance, if standard sampling is meeting the minimum requirements set by law or regulation, when a final sample is tested at the end of the line which indicates some issue with the quality and/or content of the petroleum (or tested material), local samplers along line may be initiated to provide testing of each source point. Additional samples taken may be stored in a specific canister for this purpose which may be separate from the standard sampling cans. Other items that may be modified include the size of frequency of sample pulls, the rate of pulls, etc. When a local testing is used and an impurity is found, an alarm or warning may be used to indicate such issues. This alarm may connect to an external device, may provide local noise/lights, or may have an auto shutdown. A specific customer may want the testing to continue while a warning may include information on the contents of the impurity. There may be customer-provided thresholds for various different impurities/content issues which may or may not cause a shutdown of the line. For instance, if the testing locally determines cloudy or sediment in the pipeline, this may cause the source to be shut down to avoid any potential pump issues.

The system can be understood as analogous to a typical video surveillance security system at a retail or other office space. For instance, video surveillance in such systems may take a snapshot every 5 seconds. However, once some sort of trigger is set, for instance, movement or a breach is detected, a full live recording may be taken at 30 times per second etc. Similarly, this sampling may utilize existing sample stations, but may increase the type and frequency of sampling based on some trigger.

The controller may be monitored or may provide signals via any of these industry standard means for information transfer as are known in the art, or as may be developed useful for the system. HART devices, or MODBUS, or TCP, digital, wireless, or analog signals may all be used. The behavior of the sampler, the operation of the sampler, may be controlled by a controller. Controller may take local or external commands.

Additional equipment may be added to current sampling cabinets and systems. For instance, a diverting valve may be added to a sampling system. New canisters may be used to hold additional or alternative samples. A receiver may be added to receive signals from outside source, as may include signaling devices and/or alarms. Furthermore, onboard sensors at sampling location may be used for testing. Primarily, a controller will be added or programmed for sampling. Furthermore, additional samples may be taken in triplicate, as is known in the art, to provide for neutral third party arbitration.

Controller, or processor, may be capable of storing, and communicating data regarding the attributes of testing, including quality attribute readings, the sampling methodology, etc. The controller may also be connected to a printer to print and label testing cans with quality attribute, time, location, danger of fluid, drawing info, or other useful information. Controller may also be adapted to have multiple preprogrammed thresholds of a single quality attribute. Further, controller may be capable via connection to a source or along the line to trigger a shutdown of fluid flow into pipeline stream.

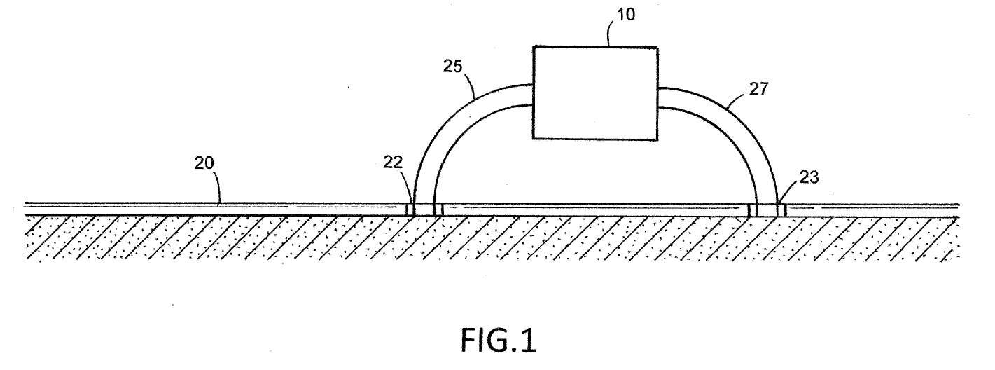

As can be seen in FIG. 1, pipeline 20 may be used to transport or otherwise facilitate the movement of a fluid 1. Fluid is preferably a petro chemical such as liquid natural gas, crude oil, etc. Pipeline 20 may be fitted with a sampling station 10. Sampling station may run a fast loop offline whereby fluid is exiting through loop-out 22 through sampling station 10 and back into pipeline via loop-in 23. Fast loop comprises loop-out 22 through initial sampling pipe-in 25 which reaches sampling system 10 which can then exit sampling pipe-out 27 and rejoin main pipeline through loop-in 23. It is contemplated that a fast loop will be preferable for the sampling, however other sampling methodologies, as are known in the art, may be useful for the present invention.

The primary purpose of the sampler is to take necessary samples as may be required by law and/or customers. The secondary purpose of the sampler is to allow for samples to be taken that can be tested on-site. Sampling system 10 preferably includes a draw apparatus as is known in the art to draw samples into containers, such as cans, as are known in the art. Sample draws may be tested when moving through the system, in the garb column, on discharge line into the receivers, in the receivers, or once in alternative testing cans. Samples will be tested for various quality attributes to determine the adaptive sampling action.

As discussed above, sampler will test for quality attributes, such as density, water content, viscosity, clarity/color/optical, temperature, pressure, flashpoint, vapor pressure, sulphur content, etc. as may be known or otherwise of interest to those testing the samples. Each of these attributes may be one quality attribute of the samples. Sampling system preferably includes at least one sensor to detect at least one quality attribute. Information from the sampler as to the quality attribute will be relayed to a processor. The processor may be local or remote. The processor will compute the quality attribute and compare with a predetermined threshold. If the processor finds that a predetermined threshold, such as low density, water content too high, missing clarity, etc., the processor may indicate an aberrant sample. Once processor detects an aberrant sample, processor may transfer information to an alarm. Such alarms may include an onsite audible and/or visual alarm, or a remote radio, or electric signal to a remote identifier.

Alternatively, if processor identifies an aberrant sample, processor may modify the process, such as providing samples into a new placement can, or more preferably preparing additional samples at a higher rate. Additional, or new, tests may be run also on fluids flowing through the fast loop for issues of viscosity, flow rate in fast loop, or flow rate in pipeline, etc.

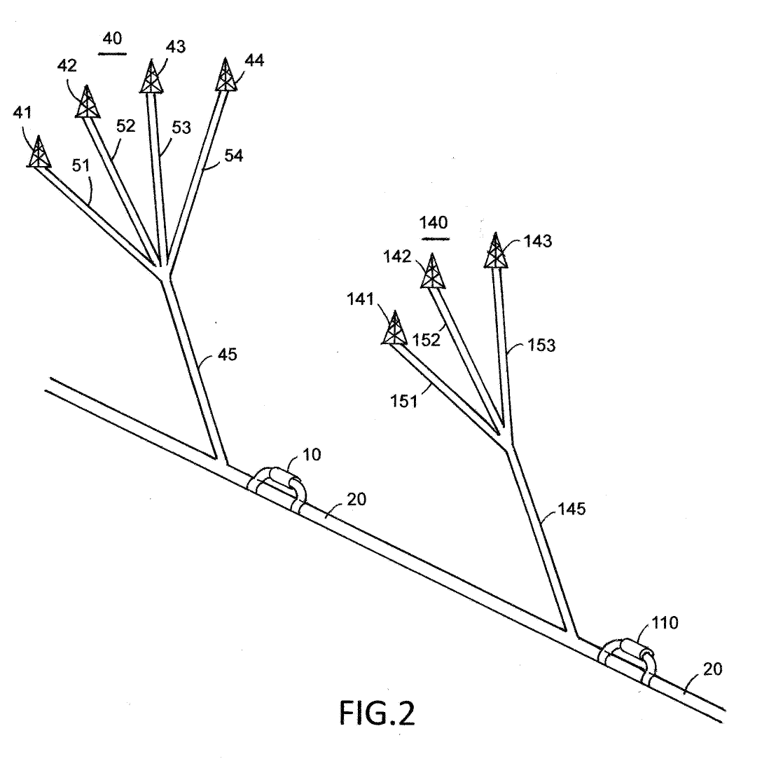

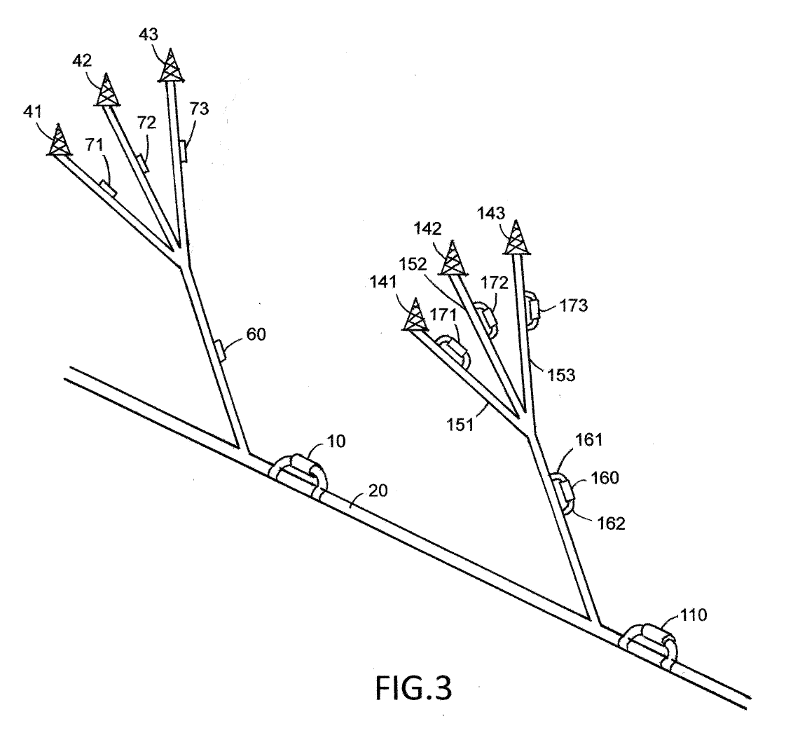

As shown in FIGS. 2 and 3, sampling stations may be set up along the pipeline. Sampling stations may be associated with one or more inlets of new product flow of fluid into the pipeline. For instance, sampling station 10 may be associated with resource field 40. Sampling station 10 will be downstream, preferably near or immediately downstream, from entry 90 into pipeline 20. Resource field 40 may include a main delivery line 45 which mates with pipeline 20 at entry point 90. Resource field 40 may include multiple drill sites, or pump sites, such as sites 41, 42, 43, and 44. Each of the sites may include its own remote supply line 51, 52, 53, and 54 that may join to source line 45.

It is contemplated that as resource fluid joins via source line 45 into pipeline 20, if the resource fluid is defective, or is otherwise aberrant such as having properties different than expected, sampling system 10 will identify a modification in the fluid properties. Once sampling station identifies an issue, sampling station may be programmed to take additional, more frequent, or alternative samplings so as to identify, confirm, or otherwise evidence the issue with the resource field. Similarly, a second resource field 140 may be included with sites 141, 142, and 143, with their own lines 151, 152, and 153, with a separate source line 145 that may enter pipeline 20 through entry point 190, whereby a second, or additional, sampling station 110 may be fitted on-line downstream of resource field 140 and entry point 190.

In alternative embodiments, as are shown in FIG. 3, additional sampling stations may be set along source lines or site lines. For instance, pipeline 20 may include sampling station 10 immediately downstream entry point 90 from resource field 40, and sampling station 110 downstream from entry point 190 from field 140. Additionally, source line 45 may be outfitted with source line sampling station 60 and source line 145 may be outfitted with sampling station 160. Sampling stations may be of any type as is known in the art or applicable for such testing, including fast loop, on-line systems, etc. Field 40 may include sites 41, 42, and 43 with site line testers (such as samplers) 71, 72, and 73, respectively. Similarly, sites 141, 142, and 143 may be fitted with testing systems (such as samplers or site line testers) 171, 172, and 173, along lines 151, 152, and 153, respectively.

The present invention may be used with any fluid flows, including liquid gas, or otherwise. Preferably, the system is used with a hydrocarbon product in liquid form, such as liquid natural gas, etc. Samplers may be set along the pipeline to test the fluid. Properties of a fluid may be determined through draws that are either moving or stationary, such as in sampled into receivers. Typically, a predetermined testing program is set whereby tests will be taken of a set amount of draw volume over a set amount of time as may be sampled into a single can as is known in the art. The predetermined draw of hydrocarbon may be tested by one or more detector, monitor, or analyzer, as is known the art. Tests requiring stationary samples are preferably taken in receivers, while those capable of being taken along a moving fluid may be taken in line. Typical gas chromatographers may be set along the flow line to capture the flow and read H2S, CO2, total sulphur content, fugitive emissions, or otherwise as would be known in the art. Preferably detectors within the sampler may be set along the intake line, the outflow line, in the grabber column, or within tubing moving fluid into receivers. The detector may be coupled to the receiver to test a quality attribute. The controller, or a processor, may be used to determine whether or not the sample meets the appropriate requirements.

In the present disclosure, the term “sampler” may be used to generally refer to a sampling system that can pull draws, and/or test fluid flowing in the pipeline. The detector may include any system capable of determining a quality attribute of the fluid. Similarly, the term “controller” can be used to describe a processor, a computer, or any system capable of receiving, handling, analyzing, and/or comparing input data with set predetermined data points. Similarly, controller send signal directly or indirectly modify sampling method or alarm. The signal is received into the controller indicating a quality attribute. A predetermined algorithm, or set of thresholds for any particular quality attribute, may be used to determine whether or not the sampling may be modified. Sampling may be modified depending on which quality attribute is affected, and a varied sampling methodology may be provided depending on the status of the quality attribute. For instance, when determining a high water content in the fluid, the system may be modified to draw a more frequent or larger volume sample. These more frequent samples can later be analyzed to determine the effect of source fluid on final pipeline conglomerate. Alternatively, if disqualifying content is detected, the sampling system may cause draws to be deposited into an alternative sampling can for alternative testing. It is often the case where a small section of flow may be contaminated (e.g. with water). In order to isolate the cause of the issue, multiple frequent samples may be taken. The new sampling regimen may be set for a set amount of time, for instance taking 20 samples per minute for a period often minutes (preferably into a separate receiver, but may also be into the standard receiver, and then returning to one sample per minute (and possibly reverting to the original receiver). The receiver holding the special regimen draws can then be used to provide the source of contamination without interrupting pipeline flow.

Further, a cloudy reading may cause the controller to alarm and/or shut down a source line flow from adding more product to the pipeline, etc. The controller should be able to modify, or change the nature of draws as is known in the art, and preferably to direct draws into sampling cans.

While sampling is not described in detail herein, one having ordinary skill in the art of pipeline samplers will understand the field of these systems, including appropriate draw rates, and should recognize an aberration in a quality attribute, as well as an alteration of sampling as described herein above. Similarly, each of the quality attributes includes many qualities known in the art for determining quality of hydrocarbon fluids, and includes many methods known in the art to test, and the system described herein can accept any testing method capable of automatically running in an isolated outpost without requiring human action on site. Remote control may be provided by a user to make a decision and cause a change in sampling, etc. While the current embodiments set forth herein are illustrative of the inventions included, these embodiments should not be understood as limiting the invention.

We claim:

1. An automated adaptive sampling system for use with a source of hydrocarbon product passing through a pipeline, said system comprising:
a. a line source of hydrocarbon fluids;
b. a sampler in fluid connection to said line source, said sampler adapted to draw a predetermined draw of hydrocarbon product from the line source as a sample, said sampler pass fluid through tubing and depositing a draw into a testing receiver;
c. a detector coupled to said testing receiver, said detector adapted to test a quality attribute of said sample either in the tubing or in the testing receiver;
d. a controller, said controller adapted to receive a remote signal indicating at least one quality attribute of said sample from the detector, said controller programmed with a predetermined algorithm for modification of sampling from a predetermined draw regimen to a new sampling regimen based on the remote signal.

2. The sampling system of claim 1 wherein said draw regimen comprises start/stop.

3. The sampling system of claim 1 wherein said draw regimen comprises a modification of frequency of samples taken.

4. The sampling system of claim 1 wherein said draw regimen comprises a modification of size of samples taken.

5. The sampling system of claim 1 wherein said draw regimen comprises a modification of rate of pull.

6. The sampling system of claim 1 wherein said system further comprises an alarm.

7. The sampling system of claim 6 wherein said alarm comprises an electronic connection to a remote external device.

8. A method of adaptive sampling a pipeline flow of petroleum-based liquid comprising the steps of:
a. connecting an inflow to extract liquid into a sampling system;
b. drawing a sample at a draw rate including a predetermined timing and a quantity;
c. testing a quality attribute of the liquid within the sampling system;
d. providing a controller in communication with the sampling system, the controller capable of receiving a information of the quality attribute;
e. receiving the information in the controller;
f. comparing the information against a predetermined quality requirement;
g. modifying the draw rate.

9. The method of claim 8 wherein said step of modifying comprises starting a sampling event.

10. The method of claim 8 wherein said step of modifying comprises reprogramming the sampling frequency.

11. The method of claim 8 wherein said step of modifying comprises reprogramming the sampling rate.

12. The method of claim 8 wherein said step of modifying comprises changing location of the sampling storage.

13. The method of claim 12 wherein further comprising the step of marking a sample draw with sample information indicating the modification of draw.

14. The method of claim 8 wherein said step of modifying comprises setting an alarm.

15. A method of preparing adaptive sampling of a fluid from a pipeline, said method comprising the steps of:
a. drawing a predetermined draw of hydrocarbon product from the pipeline as a sample;
b. depositing a draw into a testing receiver;
c. testing a quality attribute of said sample;
d. receiving a signal indicating at least one quality attribute of the sample in a controller;
e. comparing the at least one quality attribute with a predetermined quality requirement;
f. modifying the sampling regimen.

16. The method of claim 15 further comprising the step of directing further sampling to a new sampling can.

17. The method of claim 15 further comprising the step of indicating an alarm when the step of comparing indicates a deviation beyond the predetermined quality requirement.
